(19) United States
(12) Patent Application Publication
(10) Pub. No.: US 2002/0187481 A1
(43) Pub. Date: Dec. 12, 2002

(54) Methods and compositions for ultral low copy number analyte detection

(76) Inventor: Elliot R. Ramberg, Del Ray Beach, FL (US)

Correspondence Address: JOHN S. PRATT, ESQ, KILPATRICK STOCKTON, LLP, 1100 PEACHTREE STREET, SUITE 2800, ATLANTA, GA, 30309, US

(21) Appl. No.: 09/933,307

(22) Filed: Aug. 22, 2001

Related U.S. Application Data
(60) Provisional application No. 60/226,823, filed on Aug. 22, 2000.

(51) Int. Cl. C12Q 1/68; G01N 33/53

(52) U.S. Cl. 435/6; 435/7.1

(57) ABSTRACT

The present invention is directed to methods and compositions for signal amplification used in analyzing large quantities of a DNA, protein, cellular or RNA sample to detect a target analyte. The methods of the present invention, Zymogen Mediated Signal Amplification, ZMSA, minimize the occurrence of false positive and false negative conclusions in diagnostic results without DNA amplification or reverse transcriptase reactions. The signal amplification methods of the present invention employ highly increased signal production capabilities of an enzyme and its substrate. Embodiments of the present invention may take the form of an autocatalytic reaction or a synthetic enzyme.

### RELATED APPLICATIONS

This application claims priority to U.S. Provisional Application No. 60/226,823, filed on Aug. 22, 2000.

### FIELD OF THE INVENTION

The present invention comprises methods and compositions for detecting nucleic acid sequences. More particularly, the present invention comprises methods and compositions for detection and analysis of low copy number targets in a sample.

### BACKGROUND OF THE INVENTION

Diagnostics has traversed a broad range of disciplines from an initial foothold by serologic diagnostics to DNA molecular diagnostics, with PCR being the front-runner. Problems with current diagnostic technologies are that they do not enable the direct detection of species specific mRNA, species specific proteins, and also lack specificity and sensitivity. The problems of detection of molecular cancer metastasis, or residual disease, the early detection of HIV and other viral agents, sensitivity in detection of pathologic proteins or cells in normal tissue, and the need for heightened specificity and sensitivity in the determination of dysplasia, illustrate the need to provide diagnostics that will integrate nucleic acid and protein analysis into a platform that will support reliable and accurate diagnostic results. Furthermore, this technology should provide for detection of targets that are found in very small copy numbers, whether the target is low numbers of DNA, mRNA, protein and cellular targets, in the presence of a large amount of non-specific (normal) material such as genomic DNA, mRNA, protein, and cells. Additionally, diagnostic tests should be able to detect a low number of cancer cells in a mass of normal cells.

A major problem with currently available diagnostic technology has been the inability to achieve consistently high levels of specificity and sensitivity early in the infectious time-course of a disease and a similar inability to look for specific analytes at very low copy number in a large mass of non-specific analyte. An analogy for this situation would be to find a needle in a haystack. The needle may represent the specific analyte of interest whether it is DNA, mRNA, other RNA, cellular material and soluble protein. The hay would represent normal or non-target analyte of the same material.

What is needed are methods and compositions for specific and sensitive detection of analytes.

### SUMMARY

The present invention comprises methods and compositions for diagnostic applications that can analyze DNA and RNA directly without a need for DNA amplification or the need for a reverse transcriptase reaction. The methods of the present invention minimize the occurrence of false positive and false negative conclusions in diagnostic results. The methods of the present invention can also analyze cellular and soluble protein samples for low copy number targets, such as cellular or protein subpopulations. The methods of the present invention can support analysis of approximately 5,000,000 to 10,000,000 exfoliative cells or disaggregated tissue cells and detect a unique cell subset or single cell in the population. Related technologies for detecting low copy numbers of targets are found in U.S. Pat. No. 5,962,225; U.S. patent application Ser. No. 09/313,121; U.S. patent application Ser. No. 09/633,848; U.S. patent application Ser. No. 09/439,134; U.S. patent application Ser. No. 09/443,633; U.S. patent application Ser. No. 09/702,066; U.S. patent application Ser. No. 09/705,067; U.S. patent application Ser. No. 09/776,568; and U.S. Provisional Patent Application No. 60/226,823, filed Jul. 22, 2000, all of which are herein incorporated in their entireties.

These processes referred to as Haystack Processing (HP) technology include TPA, RFTA, CPA, and STM. High specificity of the diagnostic result is a characteristic of inventions comprising this technology. High sensitivity is also a characteristic of the present invention and is in part due to nature of the signal amplification processes.

The methods of the present invention comprise signal amplification methods that are used to detect ultra low numbers of any analyte, DNA, RNA, cellular material or protein. The novel signal amplification methods of the present invention are collectively referred to as Sequential Linked Enzyme Signal Amplification (SLESA). SLESA provides for a direct interaction of a number of sequential enzymes linked with each other that result in amplification of a signal from a single target from a million fold to 10 million fold or more. In other words, the presence of a single target will generate millions to multi-millions of signals that will aid in the ready detection of the presence of the very low copy target.

### BRIEF DESCRIPTION OF THE DRAWINGS

FIG. 1 is a chart showing signal generation by Zymogen Mediated Signal Amplification (ZMSA-1).

FIG. 2 is a chart showing signal generation by Zymogen Mediated Signal Amplification (ZMSA-2).

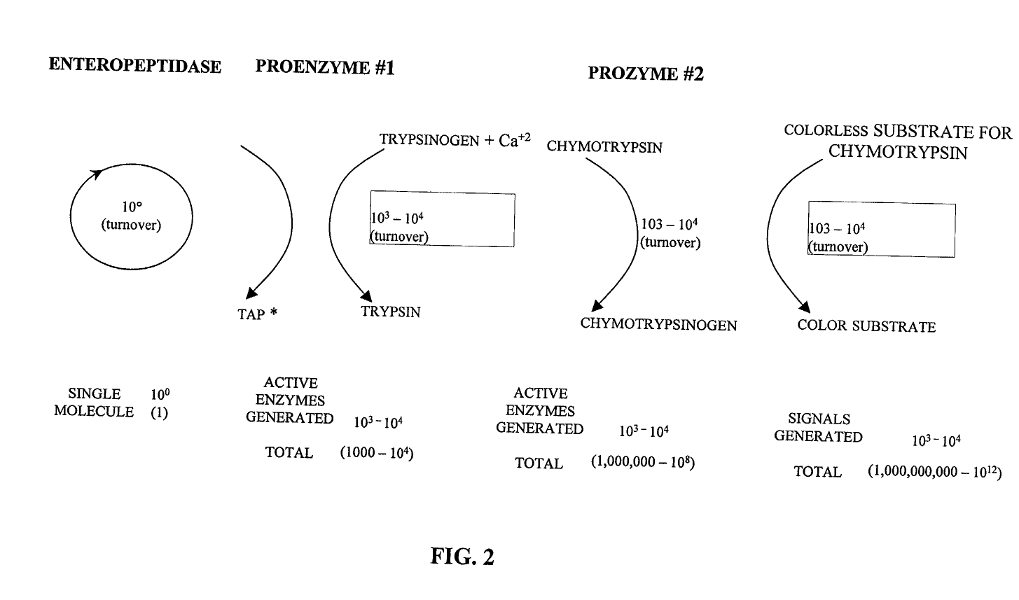

FIG. 3 is a chart showing signal generation by Autocatalytic Zymogen Mediated Signal Amplification (ZMSA-AC).

FIG. 4 is a chart showing signal generation by the ZMSA and CMSA (Complement Mediated Signal Amplification) hybrid (Z/CMSA).

FIG. 5 is an example of several zymogen cascade and zymogen conversion signal amplification reaction processes.

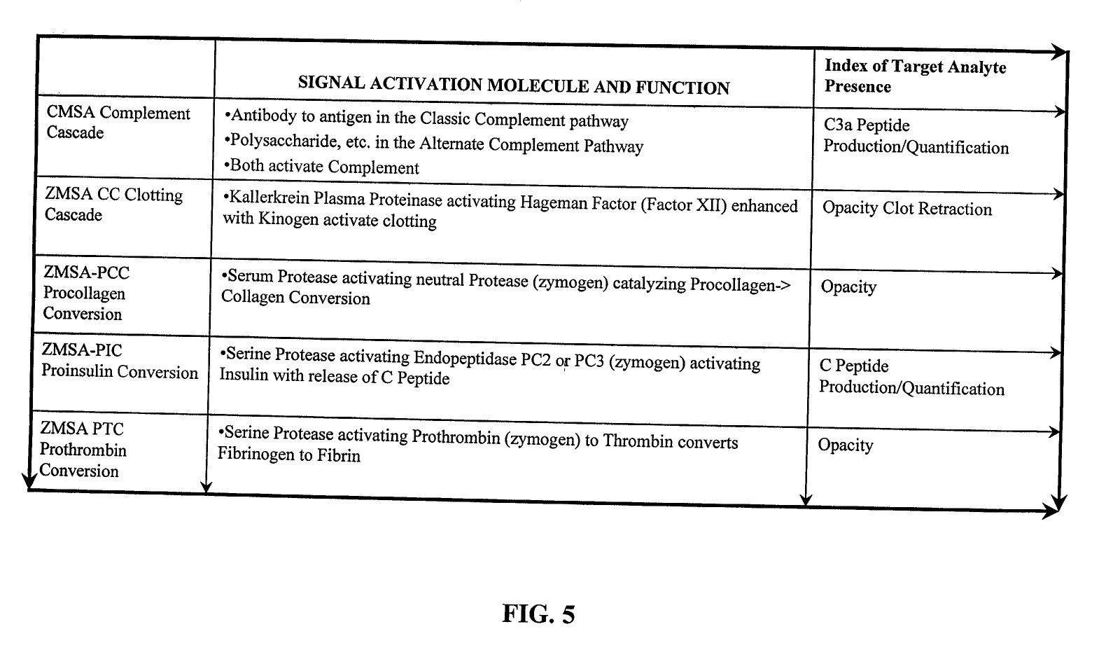

FIG. 6 is a chart showing combinations of methods for very low copy number target analyte detection.

FIG. 7 is a representation of Membrane Assisted Complement Mediated Signal Amplification technology (MACMSA) for amplification of a single immunogenic epitope on a reporter probe or a single soluble immunogen in solution.

FIG. 8 is a representation of Complement Mediated Signal Amplification as it relates to detection of single pathologic cells or nuclei in a mass of normal cells or nuclei.

FIG. 9 is a representation of a MRNA RP-TFO embodiment of SNP-TPA using a ZMSA embodiment of SLESA.

### DETAILED DESCRIPTION

The present invention comprises methods and compositions for the detection of low copy number targets. The targets comprise cellular components, DNA, RNA, other nucleic acids, synthetic and natural nucleic acids and proteins, carbohydrates, antigens and other targets. The methods and compositions of the present invention can be used, for example, in diagnostic assays to detect the presence of disease or an alteration in the physiological state or used to follow the effect of treatment agents.

The methods and compositions of the present invention are used, in particular, for assays in which non-target elimination (NTE) is accomplished. NTE minimizes the occurrence of false positive and false negative conclusions in diagnostic results and uses multiple specificity steps in the diagnostic assays. Preferably, the target or analyte is present at each step in the sample analysis to detect analyte or target. Preferably, the absence of the target or analyte at each step of the process does not result in the generation of a signal (target falsely determined present) and false detection of the target or analyte are not reported.

The STM (selective target monitoring) platform can also directly analyze large cellular and soluble protein samples for low copy number targets (cellular and protein subpopulations). STM can support analysis of approximately 5,000,000 to 10,000,000 exfoliative cells or tissue cells (disaggregated) and look for a unique cell subset or rare abundance cells in the population. STM can further support analysis of milligram quantities of soluble protein and support detection of a unique protein subpopulation with theoretical sensitivity limits to rare abundance protein molecule targets; the analogy of which is like testing the entire haystack (proteins) for a single needle (single protein molecule of interest).

The signal amplification methods of the present invention are collectively referred to as Sequential Linked Enzyme Signal Amplification (SLESA). SLESA provides for a direct interaction of a number of sequential enzymes linked with each other that result in amplification of a signal from a single target. Three embodiments of SLESA are named Zymogen Mediated Signal Amplification (ZMSA), Complement Mediated Signal Amplification (CMSA), and Membrane Associated Complement Mediated Signal Amplification (MACMSA).

A zymogen by definition is an inactive precursor that is converted to an active agent by some action, such as that of acid, alkali, or an enzyme. Herein, zymogen and proenzyme are used interchangeably. The enzyme is produced in a cell as a proenzyme (in an inactive form) to protect the cell from the activity of the active enzyme. Any molecule, such as an enzyme, that can convert a proenzyme to an active enzyme, which in turn processes a substrate, is included in this SLESA embodiment. This also includes any molecule that can generate a catalytic change in another molecule. This also includes any enzyme that can be sequentially linked to another (or others) and react with a substrate for signal amplification for target analyte detection.

Embodiments of SLESA comprise an autocatalytic reaction signal amplification (ACSA) process, where the initiating catalyst (enzyme or other molecule) is attached to the analyte (target) to be detected. An example of this embodiment is the use of nitrite ions on hemoglobin in the autocatalytic production of methemoglobin. Nitrite is singularly attached by methods known to those skilled in the art, to the target analyte, which would initiate the autocatalytic conversion of hemoglobin (reduced) to methemoglobin (oxidized) only if the target is present. In this system, the presence of methemoglobin in the sample analysis would confirm the presence of the target analyte at very low copy numbers.

Another embodiment of SLESA, synthetic polymer signal amplification (SPSA), comprises a catalytic acceleration reaction by an enzyme-like synthetic polymer, wherein a synthetic polymer possesses substrate binding moieties and adjacent catalytic moieties for its modification. When these moieties are appropriately chosen, enzyme substrate turnover rates may approach 10¹² fold. An example of such a synthetic polymer was presented by Kiefer (1972) wherein a synthetic polymer series, namely polyethyleneimine containing dodecyl (C₁₂H₂₀)—chains for binding sites and imidazole moieties for functional catalytic groups, catalyzed the hydrolysis of phenolic sulfate esters by a two-step mechanism similar to that of the corresponding natural enzyme.

In this SPSA embodiment, the polymer is conjugated to a reporter molecule by methods known to those skilled in the art, with affinity for the target analyte only. Removal of non-specific (unbound reporter), addition of the reaction product to be hydrolyzed, and detection of the hydrolysis reaction leads to the determination of the presence, and in some embodiments, the amount, of the target.

The presence of the hydrolysis product, in this example, 4-nitrocatechol can be detected by any method known to those skilled in the art to confirm the presence of the low copy number analyte. In this embodiment, a single “enzyme” (catalytic polymer) is involved in signal amplification, however, in alternate embodiments different catalytic polymers may be linked together, may be linked to enzymes, or any means known by those skilled in the art, to initiate and complete a reaction upon addition of a substrate and by analysis of the reaction product for the presence of the predicted reaction product indicative of the presence of the very low copy number analyte.

Another embodiment of SLESA called ZMSA (zymogen mediated signal amplification) involves the use of a catalytic molecule or enzyme to activate a proenzyme (inactive form) to an active enzyme, which in turn produces a predicted product upon reaction with a signal producing substrate. The zymogen cascade, once activated, can produce a very powerful signal amplification tool. The zymogen cascade is very powerful as an amplification process, due to the sequential linkage of several enzymes and their enormous substrate processing capabilities.

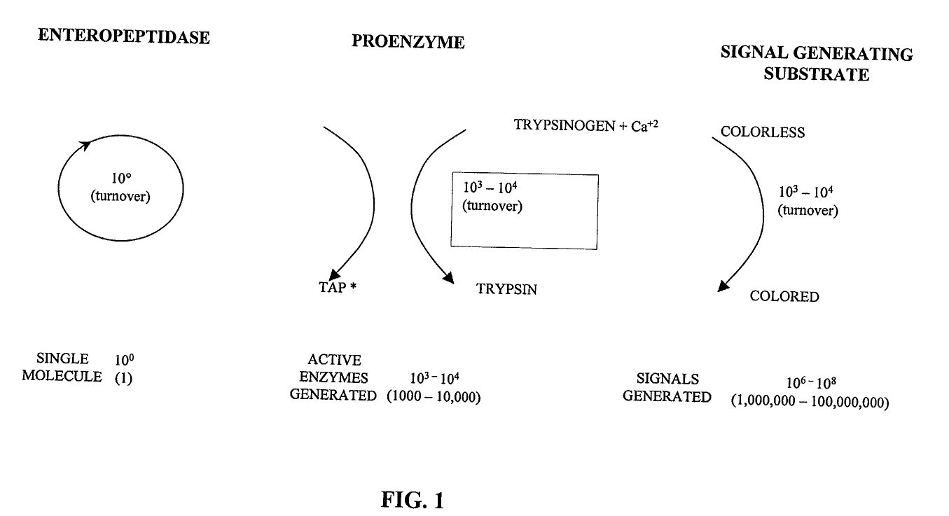

FIG. 1 represents the linkage of enzymes and the resulting substrate produced. A standard enzyme processes substrate at a rate of 3 to 4 logs per unit time (minutes.) Accordingly, linkage of two enzyme systems will provide turnover rates of 6 to 8 logs per unit time. In this example, the enteropeptidase catalytically processes trypsinogen (3-4 logs per minute turnover), which each trypsin produced catalytically processing a colorless substrate to a colored form (again at a 3-4 logs per minute turnover rate) at a total rate of 6-8 log colored substrate turnover rate.

SLESA embodiments provide a large signal amplification effect from a single target. Any sequentially linked enzyme systems known to those skilled in the art, are included in the present invention. The digestive system zymogens are representative of a small group of zymogen reactions. Other zymogen reactions include but are not limited to, enzymes in the clotting cascade, fibrinogen activation, proinsulin activation and procollagen activation. The digestive zymogen cascade and the clotting zymogen cascades present processes that support signal amplification due to the sequential linkage of several enzymes and their activities. Similarly the complement zymogen cascade affords signal amplification opportunities.

The zymogen activation reactions involving fibrinogen, proinsulin, and procollagen activation, due to the lack of extensive sequential enzyme linkage, support less significant levels of signal amplification, nevertheless valuable for signal amplification and will be referred to as zymogen conversion reactions.

The zymogen cascade and the other conversion reactions will be briefly presented to demonstrate their configuration in diagnostic testing and discuss the theoretical signal amplification levels that they will support.

Characteristics of the zymogen reaction for signal amplification (ZMSA-1):

I. The reporter probe possesses an attached activation enzyme (or other catalytic molecule).

II. The activation enzyme has as its substrate a proenzyme, 1^{st} enzyme, which it activates to active enzyme, 1^{st} enzyme.

III. A signal must be generated as a product of the active enzyme and substrate reaction, or even the product of enzyme activation of the zymogen, namely the production of the activation peptide to allow detection of the low copy number analyte.

Characteristics of the zymogen reaction for signal amplification (ZMSA-2):

I. The reporter probe possesses an attached activation enzyme (or other catalytic molecule) and has affinity only for the target analyte.

II. The activation enzyme has as its substrate, a proenzyme, 1^{st} enzyme, which it activates to active enzyme, 1^{st} enzyme.

III. The activated enzyme, 1^{st} enzyme, reacts with another proenzyme 2^{nd} enzyme, which then activates it producing active enzyme, 2^{nd} enzyme, and so on.

IV. Ultimately, a signal must be generated either as a product of an active enzyme and substrate reaction, or even a product, namely activation peptide production as a consequence of the final or any step of the sequential enzyme linked activations to allow detection of the low copy number analyte.

In ZMSA embodiments, the activating enzyme is linked to a reporter molecule with affinity only for the target analyte. Removal of non-specific (unbound) reporter, addition of proenzyme and its activation, addition of substrate, and confirmation of the presence of the reaction product resulting from substrate processing by the active zymogen by methods known to those skilled in the art will confirm the presence of the low copy number analyte. In different embodiments, one or more zymogen enzymes may be linked together and a final reaction product generated will confirm the presence of the very low copy number target present.

These embodiments are referred to as Zymogen Mediated Signal Amplification (ZMSA). The following table presents the designating nomenclature:

ZMSA DESIGNATIONS

| | No. of | No. of Enzymes Linked Including |
|---|---|---|
| Name | Proenzymes Linked | the Initial Activation Enzyme |
| ZMSA-1 | 1 | 2 |
| ZMSA-2 | 2 | 3 |

The novel signal amplification methods of the present invention comprise an increased signal due to the catalytic interaction between the enzyme and its substrate, whether it is another enzyme or a signal producing substrate.

In a preferred embodiment of ZMSA-1, an enzyme such as enteropeptidase (formerly known as enterokinase) is used to activate a proenzyme, such as trypsinogen (inactive form) to trypsin, the active form of the enzyme. The activated proenzyme, trypsin, produced in enzyme substrate amounts then reacts with substrate amounts of a non-signal producing substrate (possibly a colorless substrate in solution) that results, after reaction, in the production of substrate amounts of a readily detectable product, for example, by color.

In a preferred embodiment of ZMSA-1, a single molecule of enteropeptidase and substrate amounts of trypsin and substrate amounts of the signal producing molecule can theoretically provide as much as a six to eight log production of signal. ZMSA functions in signal amplification for the detection of analytes by placing single or multiple molecules of enteropeptidase on the analyte to be detected. The enzyme molecule is attached to a reporter moiety to for the reporter molecule. The reporter molecule comprises an enzyme and a reporter moiety that includes, but is not limited to, a reporter probe, an antibody, reporter antibody, conjugate, a reporter conjugate, binding partner pairs. The enzyme is attached to the reporter moiety by means known to those in the art for linking enzymes to other biological or synthetic molecules or polymers. The reporter molecule comprises a reporter probe, an antibody, reporter antibody, conjugate, a reporter conjugate, binding partner pairs and preferably connected to it, the reporter molecule has an inactive enzyme referred to as a proenzyme or zymogen. The reporter molecule associates with the target analyte by binding or hybridizing or other noncovalent interactions such as occur between binding partners, such as proteins, sequence recognition such as nucleic acids, or ligand or substrate interations.

ZMSA can also be similarly configured for cell and protein analysis utilizing antibodies or other molecules functioning similarly to deliver the proenzyme activation molecule to the zymogen.

The novel signal amplification method of the present invention possesses a highly increased signal production capability due to the accelerated and catalytic reaction of the enzyme with its substrate.

In a preferred embodiment of ZMSA-2, an enzyme such as enteropeptidase is used to activate a proenzyme, such as trypsinogen (inactive form) to the enzyme trypsin, the active form. The activated proenzyme, trypsin, produced in enzyme substrate amounts then reacts with and coverts a second proenzyme, namely chymotrypsinogen (inactive form) in enzyme substrate amounts to chymotrypsin (the active form) which then reacts with its specific colorless substrate to produce a color. Any signal known to those skilled in the art may be employed and any linked zymogens may be utilized.

Peptide Release in Zymogen Activation: Trypsinogen

In ZMSA-1, trypsinogen (inactive) is converted into active trypsin by removal of a hexapeptide from the n-terminal end, mediated by the action of the enzyme, enterokinase.

In a preferred embodiment molecule or molecules of enterokinase will be attached to a reporter probe with affinity for the target analyte. Capture of the target analyte and attachment to a solid surface, removal by washing of unbound reporter probe/enterokinase, addition of any zymogen (proenzyme), and addition of a signal producing substrate generating a signal upon interacting with the active enzyme results in signal detection and confirms the presence of the target analyte. In this example, the substrate for the enterokinase is the proenzyme, trypsinogen (inactive form), which is converted to trypsin (active form) with the release of the peptide. Detection of the peptide can verify the target presence. Similarly, the presence of a color product will also verify the presence of the target.

Peptide Release in Zymogen Activation: Chymotrypsinogen

In ZMSA-2, trypsinogen (inactive) is converted into active trypsin by removal of a hexapeptide from the n-terminal end, mediated by the action of the enzyme, enterokinase. In a preferred embodiment of ZMSA-2, the trypsin reacts with chymotrypsinogen (inactive) converting it into active chymotrypsin resulting in the removal of two peptide fragments (dipeptides).

In this embodiment of ZMSA-2, molecule or molecules of enterokinase are attached to a reporter probe with affinity for the target analyte. The process entails capture of the target analyte and attachment to a solid surface by any method known to those skilled in the art, hybridization of an enzyme conjugate reporter probe, removal by washing of unbound reporter probe/enterokinase, conjugate, and addition of trypsinogen, followed by addition of chymotrypsinogen, and subsequent dipeptide release. Detection of the dipeptide will verify the target presence as well as due to chymotrypsin activation. Similarly, addition of a colorless substrate, which upon reaction with chymotrypsin is cleaved and produces color also detects the presence of the low copy number analyte.

In this case enterokinase first reacts with the proenzyme, trypsinogen, which it catalytically converts to trypsin. Next the trypsin is allowed to react with a second proenzyme, chymotrypsinogen, which it catalytically converts to chymotrypsin (active). Now addition of a colorless substrate, which chymotrypsin converts to a color product, will confirm target presence. The color produced must not result from reaction of the colorless substrate with enterokinase or trypsin, the active enzymes and the color product produced must be a direct result of interaction of the colorless substrate with active chymotrypsin.

Zymogen Activation and Generation of Peptides as a Signal

In ZMSA-1 trypsinogen upon activation to trypsin releases the trypsinogen activation peptide, TAP.

In ZMSA-2 chymotrypsinogen upon activation to chymotrypsinogen by trypsin releases dipeptide fragments, chymotrypsinogen activation peptides, CAP.

It is important to note that the generation of these TAP and CAP fragments in themselves can be used as a signal. Assay for the presence of the peptides themselves are a direct indicator of the presence of the very low copy number target analyte. The peptides may be detected or quantified by methods similar to those presented for the quantification of the C3a peptide in CMSA, presented herein.

A ZMSA-1 and CMSA Hybrid Signal Amplification Assay (Complement Alternate Pathway Activation)

A preferred embodiment of ZMSA-1 results in the production of trypsin molecules. Another embodiment for single analyte signal amplification to detect the presence of these low copy number analytes is a two-step method that employs the activation of trypsin with the addition of a complement reagent. This is referred to as the Z/CMSA Hybrid Method. In this embodiment an enteropeptidase labeled probe or other reporter molecule binds to a specific target analyte. Washing to remove unbound reporter probe follows. Trypsinogen is added to be converted in enzyme substrate amounts to active trypsin. Next, complement reagent is added to the solution and via the alternative pathway the complement is activated by the cleavage of C3 into C3b with the release of the C3a peptide.

Another embodiment comprises attachment of an active protease enzyme to a reporter molecule. Hybridization of the target with the reporter probe is followed by addition of serum complement, chemically treated to block the classical pathway. The alternate pathway will allow the active enzyme to produce C3a peptides.

Another embodiment is the fusion of zymogen activation, for example, trypsinogen, and complement activation, for example where the assay is for C3a peptide production. In this hybrid embodiment, the classical pathway in the complement reagent must be blocked by an inhibitor that keeps the alternate pathway intact and available for C3a peptide generation. The trypsin, generated by zymogen activation, activates the alternate pathway and leads to the production of an amplified number of C3a peptides. A single enterokinase enzyme on a reporter probe converts enzyme substrate amounts of trypsinogen to trypsin and the trypsin produced would activate the alternate complement pathway and result in increased numbers of C3a peptide production and both participate in signal amplification by generation of millions of signals (C3a fragments) per single target analyte. These can be detected by methods known to those skilled in the art.

Another embodiment of ZMSA represents a zymogen cascade that functions in vivo and in vitro. This clotting zymogen cascade is referred to as the ZMSA-CC (clotting cascade) process. This embodiment comprises:

I. A reporter probe labeled with Kallikrin, or a molecule of identical function, which will activate the kinnin system and the remaining zymogen cascade, and initiate the formation of the fibrin clot.

II. The activation enzyme Kallikrin will cause the sequential activation of several zymogens and support an extensive amplification effect leading to the clot.

III. The result of the sequential enzyme linked signal amplification will be the formation of the fibrin clot. Two indicators of target analyte presence and subsequent clot formation are the increased opacity of the translucent plasma due to fibrin formation, and retraction of the clotted plasma from its container.

IV. Signal amplification detection.

Another embodiment of ZMSA represents a zymogen conversion that results in procollagen conversion to collagen mediated by a neutral protease enzyme (zymogen/proenzyme). This conversion reaction is referred to as ZMSA-PCC (procollagen conversion). This embodiment comprises:

I. A receptor probe labeled with a serine protease, which will catalytically activate the neutral protease, the zymogen.

II. The activation enzyme, the serine protease, activates the neutral protease to convert the soluble procollagen, clear in solution, to the insoluble fibrous collagen, forming an opaque matrix.

III. The result of neutral protease activation will be the conversion of procollagen to collagen and the formation of an opaque matrix in the reaction vessel.

IV. Signal amplification detection.

Another embodiment of ZMSA represents a zymogen conversion that results in proinsulin conversion to insulin mediated by endopeptidase PC2 or PC3 (zymogens/proenzymes). This conversion reaction is referred to as ZMSA-PIC (proinsulin conversion) process. This embodiment comprises:

I. A reporter probe labeled with a serine protease, which will activate the endopeptidases PC2 or PC3 (zymogens).

II. The activation enzyme reacts with the endopeptidases to convert the proinsulin to insulin with the release of a biologically inactive C peptide of 31 amino acid residues.

III. The result of endopeptidase PC2 or PC3 activation and proinsulin modification would be the production of the C-peptide into the solution. Quantification of C peptide production by methods known to those skilled in the art would provide an indication of the extent of zymogen conversion of proinsulin and a quantitative measure of signal amplification, indicates the presence of the low copy analyte of interest.

Another embodiment of ZMSA represents a zymogen conversion that results in fibrinogen conversion to fibrin by activated prothrombin. This conversion reaction is referred to as ZMSA-PTC (prothrombin conversion). This embodiment comprises:

I. A reporter probe labeled with a serine protease that will activate the prothrombin (inactive) zymogen to active thrombin.

II. The activation enzyme the serine protease reacts with prothrombin (proenzyme) converting the prothrombin to active thrombin, which in turn converts soluble fibrinogen to fibrin.

III. The resulting formation of the fibrin clot will be followed by its transition to an opaque and retractive clot to indicate the amplification reaction result due to the presence of the low copy number analyte.

IV. Signal amplification detection.

ZMSA can be similarly configured for cell and protein analysis utilizing antibodies or other molecules with attachment of the activation molecule functioning similarly to deliver the proenzyme activation molecule to the target. ZMSA also functions in nucleic acid analysis (mRNA and ds and ss DNA) and the oligonucleotide attached activation molecule can initiate signal production.

### ZMSA mRNA TPA EXAMPLE 1

The following Example 1 for ZMSA nucleic acid analysis utilize the RP-TFO generation of a PNAS or protected nucleic acid target sequence.

Example 1 possesses three levels of specificity, provided by, one, the exonuclease (3′→5′) degradation of the mRNA, and two, mRNA target RP-TFO capture, and three, subsequent attachment of the reporter probe (either a duplex forming oligo, or Reverse Phase-Triplex Forming Oligonucleotide).

Step I:

The RNA is prepared by methods known to those skilled in the art. RNA isolation usually provides a protein denaturation step, and treatment with a chaotropic agent (guanidinium sulfate) which denatures the environmental and cellular ribonucleases present.

Step II:

The sample RNA is hybridized with a capture RP-TFO that is biotinylated and specific for the MRNA target at pH 5.5. The RP-TFO is specific for a 12 mer polypyrimidine region with one purine insertion on the target mRNA. See related documents. If the RNA is mRNA, slight heating of the mRNA may aid in triplex formation at target site. If the RNA is rRNA, more extensive heating (˜90° C.) of the RNA will remove the secondary structure and allow the RP-TFOs to form the stable triplex at the target site. This is the first level of specificity.

Step III:

Add an exonuclease (3′→5′) to degrade all non-specific mRNA and target mRNA only from the 3′ end to the capture RP-TFO. The capture RP-TFO provides a PNAS which renders the target nuclease resistant. The enzyme must possess enough activity at the pH selected for use, preferably 7.2-7.6 or lower, if enzyme activity allows to degrade non-specific mRNA. At this point, the target/capture probe complex forms the PNAS (protected nucleic acid sequence). A single stranded mRNA nuclease (3′→5′activity) according to classical TPA is now added to destroy all non-specific mRNA targets, degrade them in a 3′ to 5′ direction and also the 3′ end of the target analyte. The RP-TFO will protect the MRNA target analyte from the RP-TFO binding site to the 5′ end of the mRNA target analyte from exonuclease degradation. The reporter probe, thus as a duplex or triplex, will be placed on the 5′ end of the target analyte mRNA (between the RP-TFO capture probe and the 5′ end of the mRNA target). This is the second level of specificity.

It is preferred in these methods that the assay pH remain as low as possible to generate the most stable PNAS with the RP-TFO, and prevent environmental ribonuclease assay interference, due to the fact that these have no activity below pH 7.0.

Step IV:

Streptavidin coated magnetic beads are added to the enzyme treated sample and bind the mRNA target with its attached capture biotinylated RP-TFO

Step V:

The magnetic beads are washed to remove non-specific material with buffer at preferably, pH 7.2.

Step VI:

The magnetic beads are next hybridized with a reporter probe either a duplex forming oligonucleotide or a triplex forming RP-TFO both possessing the zymogen activation molecule. This is the third level of specificity of the assay.

Step VII:

The magnetic beads are washed to remove unbound reporter probe with buffer, preferably at pH 7.2.

Step VIII:

Zymogen is added to the magnetic beads with the attached PNAS containing the activation enzyme or molecule at the appropriate reaction pH.

Step IX:

Depending on the use of the ZMSA 1 or ZMSA 2 assay formats, add substrate for the active zymogen, or a second zymogen followed by its substrate are added at the appropriate pH for substrate conversions to a detectable form. Known detection methods include chemiluminescent, calorimetric, fluorescence or other signal detections known to those skilled in the art.

Step X:

Detect the signal.

### ZMSA mRNA TPA EXAMPLE 2

The following Example 2 utilizes a single reporter RP-TFO possessing the zymogen activation molecule. This assay possesses only one level of specificity, appropriate for diagnostic detection of greater number of abundance targets. This one level is based on the hybridization of the mRNA target with the reporter RP-TFO.

Step I:

The RNA is prepared by methods known to those skilled in the art. RNA isolation usually provides a protein denaturation step, and treatment with a chaotropic agent (guanidinium sulfate) which denatures the environmental and cellular ribonucleases present. Care must also be taken to remove carbohydrates, glycoproteins and any other non-specific molecule possessing a cis-diol moiety that will react with the phenylboronic acid coated magnetic bead and thereby compete with the ribose of the RNA species present. The RNA must be heated to remove secondary structure (˜90° C.) and a labeled reporter RP-TFO is added. The label may be any known to those skilled in the art. The RNA sample is then cooled slowly to permit triplex formation and generation of PNAS.

Step II:

Phenylboronic coated magnetic beads are added to the RNA preparation and all RNA species present (mRNA, rRNA, and tRNA) from ionic interactions via their ribose sugar with the magnetic bead coating (deoxyribose of DNA does not react) under conditions known to those skilled in the art. Again the strategy is to keep the pH low, however the boronic acid linkage interaction occurs optimally at pH 7.2-7.6.

Step III:

Wash the magnetic beads to remove non-RNA materials with buffer at the appropriate pH, keeping the pH as low as possible.

Step IV:

Zymogen is added to the magnetic beads with the attached PNAS containing the activation enzyme or molecule at the appropriate pH, preferably7.2-7.6.

Step V:

Depending on the use of the ZMSA 1 or ZMSA 2 assay formats, add substrate for the active zymogen, or a second zymogen followed by its substrate are added at the appropriate pH for substrate conversions to a detectable form, for example, chemiluminescent, calorimetric, fluorescence or other signal detection type known to those skilled in the art.

Step VI:

Detect the signal.

A preferred embodiment of ZMSA ds DNA TPA comprises isolating genomic DNA. The undenatured genomic DNA is heated (to˜55° C.) to allow it to “breathe” and two RP-TFOs are added. One RP-TFO or both may have capture molecules (ex. biotin) to attach the RP-TFO/DNA complex, the PNAS, to a solid support (ex. streptavidin coated magnetic bead). The RP-TFOs forms a triplex with the target sequence, called the PNAS. Each RP-TFO targets regions on opposite strands of the genomic target DNA protecting the PNAS from EXO III degradation, though same side targeting may also be effective. Exonuclease III and a restriction endonuclease with no restriction site between the RP-TFOs are added and incubated. The restriction sites are preferably located upstream and downstream from the target. The PNAS is captured and attached to a solid support, washed and a duplex reporter probe, which in one embodiment possesses the c-myc peptide, is hybridized to a region between the two RP-TFOs to either strand. An IgG anti c-myc/enterokinase conjugate is added to attach the zymogen activation enzyme only to the reporter probe on the target analyte via the c-myc/anti c-myc interaction. The PNAS is washed and zymogen is added and incubated. The PNAS is again washed and the colorless substrate is added and incubated. The generation of a colored moiety after enzyme and substrate interaction is indicative of target analyte presence.

A preferred method of the present invention for detecting a target analyte, comprises:

hybridizing the target analyte in a solution specific for hybridizing the target analyte with at least one oligonucleotide, wherein the oligonucleotide comprises an affinity molecule that binds to a solid substrate having the other molecule of the affinity pair;

capturing the target analyte;

hybridizing to the target analyte a reporter probe to form a complex;

adding an enzyme conjugate for attaching an enzyme to the complex under conditions allowing for activation of a proenzyme by the enzyme conjugate; and

activating the proenzyme,

wherein the activated proenzyme reacts with a non-signal producing substrate,

wherein the interaction with the non-signal producing substrate resulting in the production of a detectable and quantifiable signal indicates the presence of the target.

The novel signal amplification power of a zymogen cascade is a result of enhanced signal production capability of the cascade and its products. This is depicted in Table 1.

Table 1 presents the enhanced production of the C3a peptide in the complement zymogen cascade, referred to as CMSA (Complement Mediated Signal Amplification). What is shown is an amplification of C3a peptides produced by presence of the low copy number target analyte, complement fixation, and activation via the Classical Complement Pathway, a trend also observed due to target analyte presence, and the activation of complement via the Alternate Complement Pathway.

TABLE 1
INACTIVE COMPLEMENT PEPTIDE (ICP)
CHARACTERIZATION AND QUANTIFICATION IN CMSA

| | | Number ICPs produced |
|---|---|---|
| 10³ sites of fix C' per | Nature of | based of binding of each |
| membrane surface | ICP generated | C1s molecule |
| (First Amplication Step) | | |
| C1q, C1r, C1s | NONE | NONE |
| C4b | C4a | 10³/cell or nuclear |
| | | membrane |
| C4b2b3b | C3a | 10³/cell or nuclear |
| | | membrane |
| C4b2b3b5b | C5a | 10³/cell or nuclear |
| | | membrane |
| C6, 7, 8, 9 | NONE | NONE |
| (Second Amplification | 200 fold increase | |
| Step) | in ICPs | |
| C1q, C1r, C1s | NONE | NONE |
| C4b | C4a | 200 × 10³ |
| | | 2 × 10⁵ per C1s bound |
| C4b2b3b | C3a | 2 × 10⁵ |
| C4b2b3b5b | C5a | 2 × 10⁵ |
| C6, 7, 8, 9 | NONE | NONE |
| (Third Amplification | 200 fold increase | |
| Step) | in ICPs | |
| C1q, C1r, C1s | NONE | NONE |
| C4b | NONE | NONE |
| C4b2b3b | C3a | 200 × 2 × 10⁵ or |
| | | 4 × 10⁷ or 40 Million |
| C4b2b3b5b | C5a | Not amplified here (same |
| | | value as amplification |
| | | step number 2) NONE |
| C6, 7, 8, 9 | NONE | NONE |

SUMMARY: ICP numbers produced in CMSA based on the presence of a single pathologic target cell
Primary Amplification Step 3 × 10³ ICPs
Secondary Amplification Step 6 × 10⁵ ICPs
Tertiary Amplification Step 4 × 10⁷ ICPs
Total ICPs generated per single pathologic target 4.0603 × 10⁷ ICPs 40 Million

Zymogen/enzyme systems can also be used in Selective Target Monitoring Platform assays where the molecule attachment is to a cell subset is mediated by attachment to a monoclonal or other antibody molecule with specificity to any surface antigen on the cell subset of interest that has the activation enzyme conjugated to it. This enzyme molecule attachment in the Selective Target Monitoring Platform assays could be attached to a unique soluble protein molecule by similar attachment to an enzyme conjugated IgG or other antibody molecule with specificity for an antigenic determinant on the protein molecule or other soluble immunogen by any other method to achieve the same known by those skilled in the art.

The following are probe based ZMSA signal amplification methods for target detection in analyses using mRNA RP-TFO TPA, and DNA RP-TFO TPA. disclosed in the previously cited patents and patent applications.

ZMSA can also be similarly configured for cell and protein analysis utilizing antibodies or other molecules functioning similarly to either deliver the proenzyme activation molecule to the zymogen or by the use of membrane assisted CMSA and C3a quantification.

mRNA TPA Using the RP-TFO and ZMSA Embodiment of SLESA and Automation Thereof

The present invention comprises n embodiment of mRNA RP-TFO TPA wherein RNA and DNA are simultaneously precipitated in a sample and the MRNA is differentially removed with a poly dT coated magnetic bead, and the DNA is removed by use of a magnetic bead with a coating characterized by a high net positive charge.

Step I:

The mRNA is isolated by methods known to those skilled in the art. Milligram quantities can be simultaneously assayed. This can be achieved by use of the AGENDA (Automated Genomic Diagnostic Analyzer, CyGene, Inc., Sunrise, Fla.) by the following steps.

Blood or other sample is collected and the cells are lysed in a microtiter plate well by any known method, such as with detergent. The sample is treated to, degrade and precipitate protein, such as nucleases, using for example SDS/KCL, proteinase K treatment, and guanidinium chloride treatment leaving the cellular DNA and RNA in solution.

The DNA and RNA are differentially removed from the deproteinated cell lysate in the well. Removal may be by attachment to a solid substrate, for example, the mRNA is selectively removed by movement of the lysate to a microtiter plate well containing poly dT coated magnetic beads which have affinity for the poly A 3′ mRNA regions. The beads are removed and placed in another microtiter plate well for washing. The DNA can be selectively removed from the remaining lysate, for example, by addition to the lysate of magnetic bead with a net positively charged surface with affinity for the highly negatively charged DNA molecule after removal of the mRNA, which are then removed to another microtiter plate well for washing.

Step II:

The mRNA is hybridized to the poly dT coated magnetic bead. The bead, after washing, is transferred to another well of a microtiter plate where the mRNA is dissociated from the bead using heat, alkali, or any other method.

Step III:

Hybridize the nucleic acid in solution specific for the mRNA molecules of the analyte or target of interest with an RP-TFO (Reverse Phase-Triplex Forming Oligonucleotide). The RP-TFO possesses an affinity molecule (for example, a biotin that binds to a streptavidin coated magnetic bead). This is a capture reverse polarity oligonucleotide to attach the specific target analyte mRNA to a solid support. This is the first level of specificity.

Step IV:

A single stranded 3′→5′ nuclease is optionally added to the well containing the beads to degrade any non-specific non-target mRNA remaining and specific target mRNA from the poly A 3′ end up to the RP-TFO anchor/attachment site, but not between the RP-TFO attachment site and the 5′ end of the mRNA target analyte. This is the second level of specificity.

Step V:

Next a magnetic bead coated with streptavidin (or any other molecule of an affinity pair) is added and allows capture of the mRNAs of interest through the biotin conjugated RP-TFO that attaches the specific target mRNA to a solid support, preferably at a pH range of 4.5-7.5.

Step VI:

The beads are removed to a new microtiter plate well for washing and removal of non-specific non-target material (other mRNA molecules).

Step VII:

A reporter probe (which may be an 10 mer) is designed and is added to the well and hybridized to the 5′ end of the mRNA target to form a duplex. Another embodiment provides for another RP-TFO to be used, assuming the availability of a 10 mer poly rich region on the 5′ end of the target.

This reporter probe may carry a mediator, such as a c-myc peptide, which is added during oligonucleotide synthesis of the probe at a specific site to assist with binding of the reporter conjugate or is the first step in signal generation, namely the IgG anti c-myc alkaline phosphatase conjugate.

Step VIII:

Wash the magnetic beads to remove unbound reporter probe and place the beads in another microtiter plate well with buffer at low pH.

Step IX:

Add an enzyme conjugate, such as IgG anti c-myc-enteropeptidase, to attach the enteropeptidase to the PNAS (protected nucleic acid sequence) at lowest pH that will accommodate an antigen/antibody interaction.

Step X:

Remove the magnetic beads and wash to remove excess unbound conjugate with buffer, preferably at the low pH 6.5-7.2.

Step XI:

Place the magnetic beads in another microtiter plate well in buffers. Under conditions allowing for activation of trypsinogen (a proenzyme) for example, Ca⁺⁺ (20 mM) is added and the pH is maintained at 7.6-8.0; also substrate amounts of the proenzyme trypsinogen is added. Use of buffers at pH 7.6 will require adoption of extensive measures to inactivate all environmental ribonuclease molecules.

Resolution to the high pH problem can be resolved, thusly. If human trypsinogen 1 and 2 are used, the following is known by those skilled in the art. The activation of human trypsinogens 1 and 2 by porcine enterokinase at pH 5.6 shows that the two human zymogens are equivalent substrates for this enzyme and that both proteins are activated faster than the cationic bovine trypsinogen. At pH 8.0 and in the presence of 20 mM calcium the two human trypsinogens are activated by either human trypsin at the same rate but the affinity of both trypsins is higher for trypsinogen 1 than for trypsinogen 2. Two Ca2+ binding sites are identified in the two human zymogens and their pK(Ca2+) values determined. For trypsinogen 1 the values are respectively of 2.8 and 3.3 for the primary and secondary Ca2+ binding sites, and for trypsinogen 2 or 3.4 and 2.7. These values are markedly different from those obtained for bovine cationic trypsinogen, especially in the case of trypsinogen 1. These results point out a different degree of saturation of the calcium binding sites of the 2 human zymogens that must exist in physiological conditions, suggesting different biological activities of the two trypsinogens.

Enterokinase (Enteropeptidase) pH 5.6 also activates human trypsinogens 1 and 2 and reacts faster with them than with cationic bovine trypsinogen.

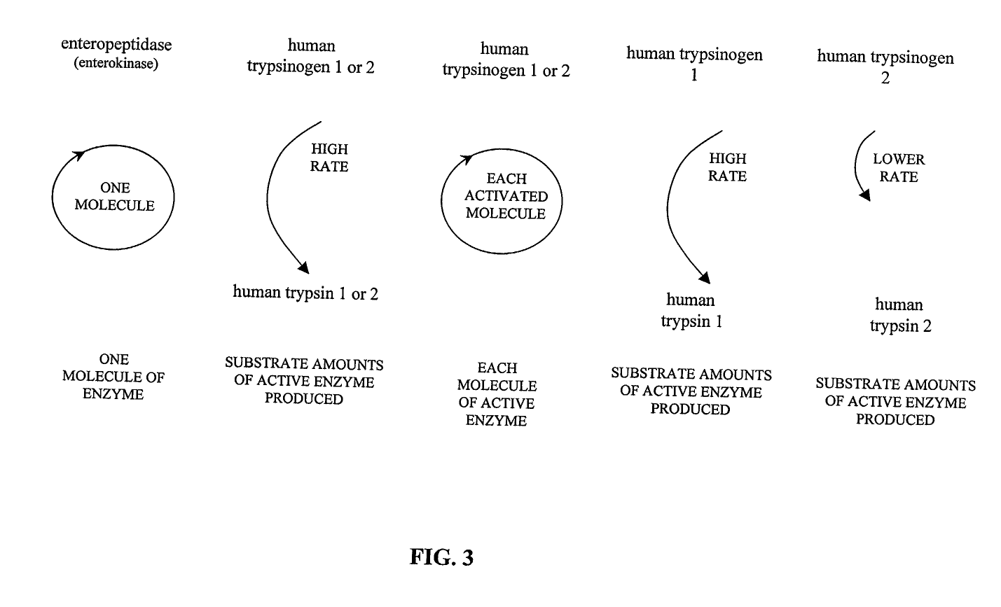

At pH 8.0 and 20 mM Ca⁺² the two human trypsinogens are activated by either human trypsin at the same rate but the affinity of both trypsins is higher for human trypsinogen 1 than human trypsinogen 2. This supports the autocatalytic reaction. This is illustrated in FIG. 3, which shows the autocatalytic effect of the conversion using the enteropeptide and human trypsinogen.

Step XII:

Incubate the PNAS/enteropeptidase conjugate complex and the trypsinogen for optimal time and temperature. The trypsinogen is converted to its active form trypsin in substrate amounts (thousands of enzyme molecules) at the lowest pH that will permit zymogen activation.

Step XIII:

Add a substrate that upon cleavage by trypsin will generate a molecule that is colored, fluorescent, chemiluminescent, or other ways detectable and monitor the production and quantify. If desired. It is important to note that any detection substrate may be used, and that activation of the proenzyme, namely to trypsin can as well be monitored by assays for the production of the Trypsinogen Activation Peptide (TAP) by any method known by those skilled in the art.

Interestingly the proenzyme trypsinogen, used in ZMSA embodiments of SLESA, and complement, used in CMSA and MACMSA, crossover and interact. It is known by those skilled in the art that trypsin will activate C3 in the alternate complement pathway and result in the production of increased numbers of C3a peptides.

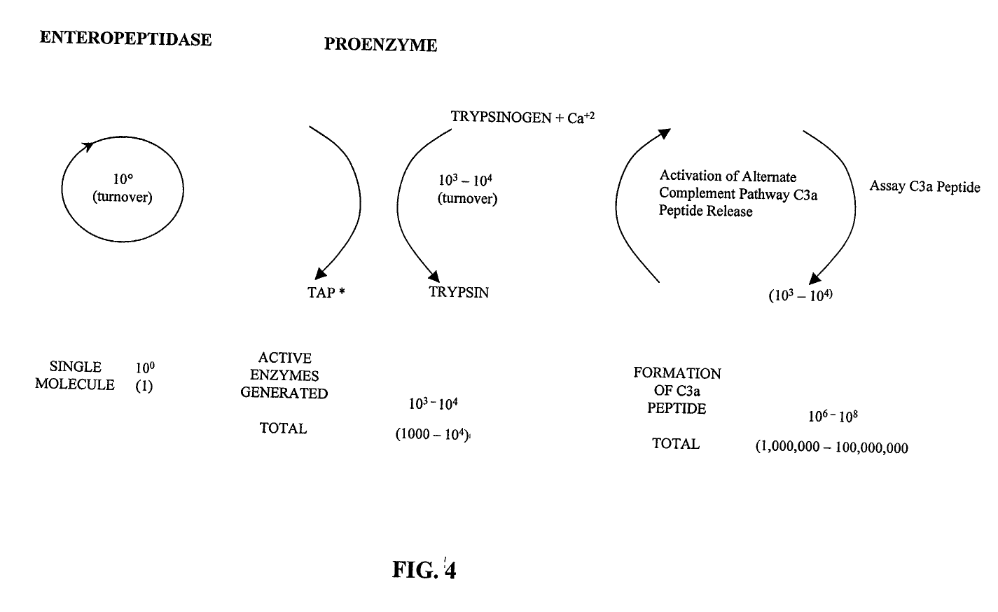

A ZMSA/CMSA assay (Z/CMSA) comprises an activated proenzyme (such as, trypsinogen) which uses complement as a signal amplification substrate followed by quantification of C3a production. This is depicted in FIG. 4.

DNA TPA Using the RP-TFO and ZMSA Embodiment of SLESA and Automation Thereof

Step I:

The DNA is isolated by methods known to those skilled in the art. Milligram quantities can be simultaneously assayed. This can be achieved by use of the AGENDA (Automated Genomic Diagnostic Analyzer of CyGene) by the following steps.

DNA targets allow pH limits to be less of an issue due to the lack of effect of environmental nucleases, but preferably uses pH at which an RP-TFO can form a triplex PNAS, usually pH range 4.5-7.0 (most stable at lower end pH).

Once the DNA is isolated from the sample, for example, as detailed above,

Step II:

The DNA, isolated on the positively charged magnetic bead, is placed in another plate well and is then heated to 40°-50° C. or another temperature that allows the DNA to “breathe” and partially separate both strands and allow the substituted purine region of the RP-TFO pair to compete for the Crick (polypyrimidine) strand of the target (same or opposite strands), with each binding to a different site both upstream and downstream from the target at a pH in the range of 4.5-6.0 (most stable triplex formation).

Step III:

Add two RP-TFOs with an affinity capture molecule (a biotin) on one or both. Two RP-TFOs are preferred, one specific for a site upstream from the target and one specific for a site downstream from the target.

The modified purine bases in the RP-TFO will allow the RP-TFOs (2) to displace one strand of the relaxed target DNA thereby separating the Watson and Crick strands of the target in order to accommodate binding of the c-myc substituted reporter probe (forming a duplex or triplex with either strand of the target) between the two RP-TFO sites. Preferably, the RP-TFO forms a pyrimidine—purine—pyrimidine triplex, which with its substituted purines forms the most stable triplex (more stable than a duplex structure) as determined by melting point determinations and circular dichroism studies. This is the first level of specificity.

Step IV:

Add EXO III to degrade non-specific DNA and a restriction endonuclease that has no restriction site located between the two RP-TFOs but possesses sites both upstream and downstream from the target to degrade all non-specific DNA and incubate at optimum time and temperature.

The is the second level of specificity.

Step V:

Add a magnetic bead to the well that is coated with the other affinity molecule (streptavidin) of the pair to capture the RP-TFO attached target. The affinity remains intact in the pH range of 6.0-7.5.

Step VI:

Remove the magnetic beads to another plate well and wash to remove non-specific DNA and protein (pH 4.5-6.0).

Step VII:

Place in another well to which is added a reporter probe (which may also be an 10 mer) which hybridizes to the target region of the dsDNA (either strand) to form a duplex (another embodiment allows another RP-TFO to be used, assuming the availability of the 12 mer poly rich region the target).

This reporter probe may carry a mediator, such as a c-myc molecule, which is added during synthesis of the probe at a specific site to initiate signal amplification. Incubate at optimal time and temperature at a pH range from 4.5-7.5.

Step VIII:

Transfer the magnetic beads to a well containing buffer and wash the magnetic beads to remove unbound reporter probe in buffer at pH 4.5-7.5.

Step X:

Transfer the magnetic beads to a new well and add an IgG anti c-myc-enteropeptidase enzyme conjugate to attach the enteropeptidase to the PNAS for subsequent signal generation again at any pH, that will support antigen/antibody interactions and activation of the proenzyme.

Step X:

Wash to remove excess unbound conjugate at the same pH.

Step XI:

Transfer the beads to a plate well and add trypsinogen under conditions allowing for its activation to trypsin. For example, Ca⁺⁺ (20 mM) is added and the pH is maintained at 7.6-8.0; and the PNAS/conjugate complex and the proenzyme is incubated at optimum time and temperature for activation of the proenzyme. A preferred embodiment employs human trypsinogens 1 and 2 (previously described herein). The proenzyme is converted to its active form trypsin in substrate amounts (thousands of enzyme molecules).

Step XII:

Add a substrate to the well that upon cleavage by trypsin will generate a molecule that is colored, fluorescent, chemiluminescent, or other ways detectable and monitor the production and quantify spectrophotometrically at the appropriate wavelength.

It is important to note that any detection substrate may be used, and that activation of the proenzyme, namely to trypsin can also be monitored by assays for the production of the Trypsinogen Activation Peptide (TAP) by any method known by those skilled in the art.

SLESA as it Relates to all Diagnostic Applications

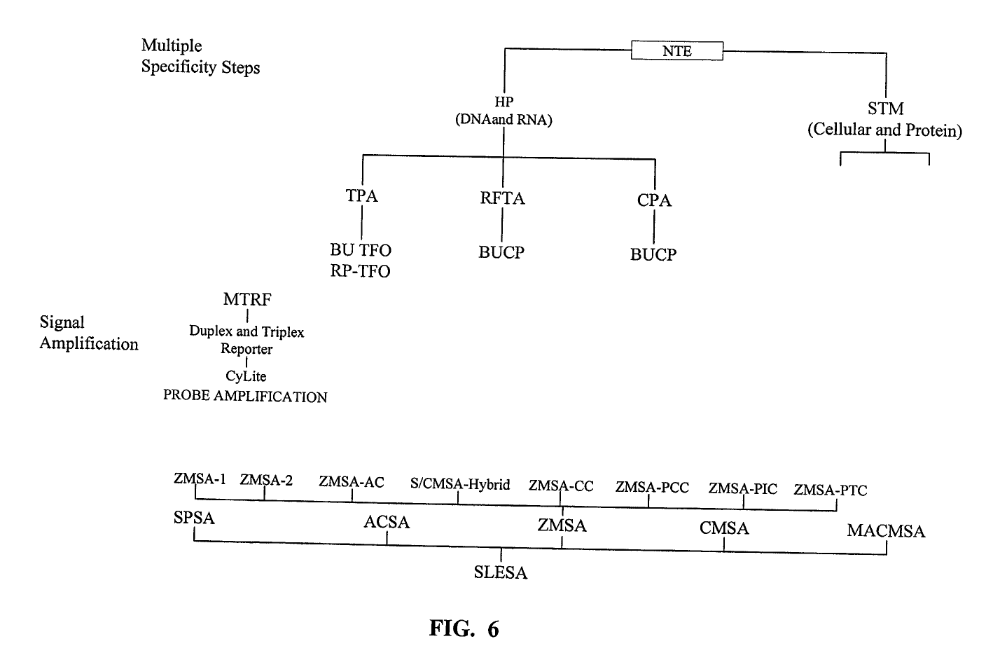

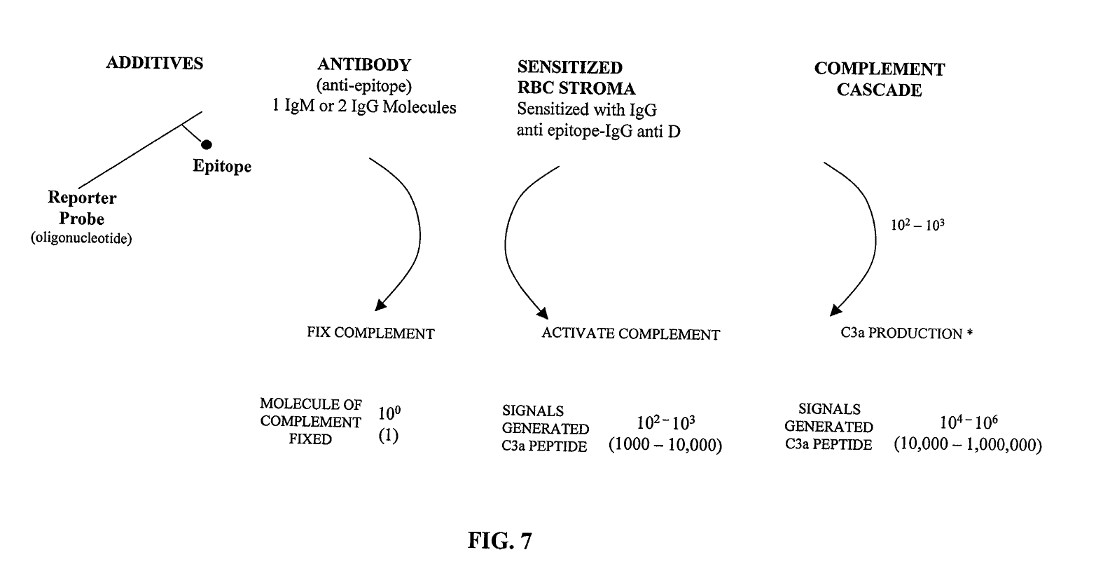

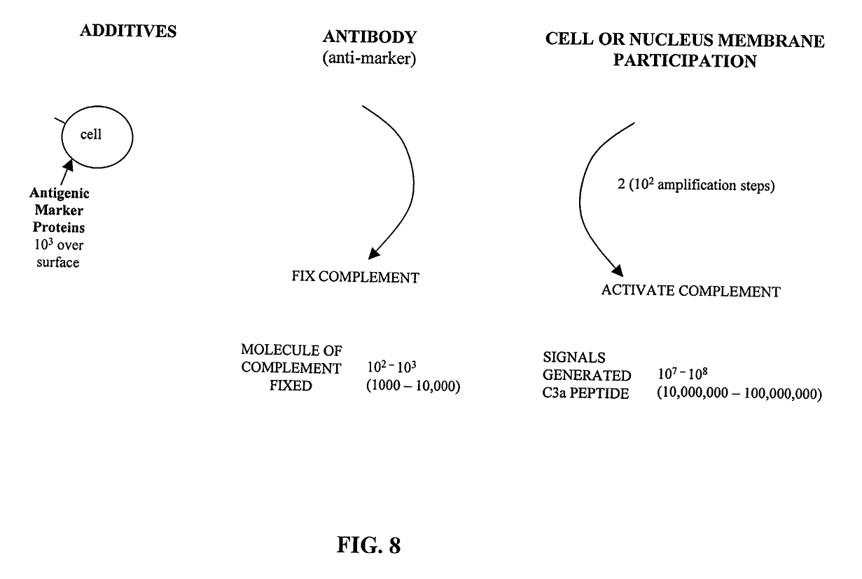

SLESA interacts with multiple assay formats and FIG. 6 depicts the manner with which the signal amplification technology interfaces with it. The signal generated by Complement Mediated Signal Amplification (CMSA) is depicted in FIG. 8, and the Membrane Assisted Complement Mediated Signal Amplification (MACMSA) is depicted in FIG. 7, are also embodiments of SLESA.

Diagnostics must be also capable of functioning at supramolecular levels to confirm the presence of the pathologic or other cellular target in tissues, especially if there is a low copy number of target. STM functions on a cellular or nuclear level to negate the presence of normal cells or nuclei in the sample, by the generation of no signal from normal cells or nuclei, while supporting the extensive generation of signals only from the presence of the pathologic cell or nucleus.

STM on a cellular level is monitored by complement mediated signal amplification or CMSA. CMSA is based on the fixation and activation of complement by interactions between cell subset specific surface membrane proteins, and monoclonal or other antibodies, specific to these, that initiate the complement fixation process. CMSA is an embodiment of SLESA due to the fact that the complement cascade is, indeed, itself a series of selective linked enzymes (an enzyme cascade).

CMSA, see FIG. 8, is used for detection of target cells and supports nonspecific target elimination (NTE) in any biological sample including:

all body fluids;

disaggregated cells suspended in a buffer solution, such as lymph nodes and fine needle aspirates;

environmental samples.

Soluble protein (peptide) pathologic or other targets can also be analyzed by STM on a soluble protein molecular level that is monitored by use of membrane associated complement mediated signal amplification or MACMSA,_see FIG. 7. This can be achieved in samples of any body fluid, cell cytoplasm, nucleoplasm, or any liquid sample, for example, environmental.

Another function of MACMSA is to detect and monitor non-protein chemicals in solution that share some of the common characteristics with peptides or proteins, namely, of being immunogenic thereby fixing and activating complement via the classical pathway, and of containing polysaccharides or other related molecules that activate complement (CMSA) via the alternative pathway, both known to those skilled in the art to activate the zymogen cascade.

MACMSA is used for detection of appropriate soluble target molecules and supports NTE in any biological or environmental fluid sample including:

all body fluids,

any protein (soluble) fluid suspension,

environmental fluids,

chemical and material processing fluids containing the soluble target.

Unique pathologic proteins or other immunogens at low molecule number in a vast excess of normal proteins are identified using STM and MACMSA, with high specificity and sensitivity. The specificity is insured by the presence of multiple specificity steps, and the sensitivity is supported by the minimization of signal background by non-specific target elimination in the samples (extracellular or intracellular), and generation of signal from all pathologic target molecules either intracellular in the cytosol or of exogenous pathologic target. This is achieved in NTE by eliminating the generation of non-specific signal from normal material and detecting the presence of the pathologic immunogen by signal emanating from the analyte of interest only.

Another embodiment of NTE utilizes Selective Target Monitoring (STM) to selectively capture the cell population or cell subset of interest, and selectively lyse (withheld lysis) only those cells of interest to analyze them and their contents for cell surface disease markers, and provide analysis of their nucleic acid targets. STM reduces the deleterious effect of the contents of non-specific normal cells and their nucleic acid or protein content on the diagnostic assay in the search for a pathologic target.

MACMSA an Embodiment of SLESA for Ultra Low Nucleic Acid Target Detection

Configuration of signal amplification involving nucleic acid targets is important in target detection on the molecular level for achieving single analyte copy recognition. MACMSA is used at the molecular level by using compositions comprising attachment of an antigenic epitope or a peptide comprised of numerous epitopes to an oligonucleotide which would act as a reporter probe. One embodiment of MACMSA comprises using a single immunogenic epitope to produce increased numbers of C3a molecules upon fixation and activation of complement via the classical complement pathway.

The extent of complement fixation and activation is influenced by many factors, these include:

Avidity of the epitope and monoclonal antibody,

Concentration of key intermediates in the complement cascade. For example, spiking native complement with additional C3 will increase the numbers of C3a produced by the presence of a single target analyte and reporter epitope in the assay,

Method of complement fixation either by the classical pathway or the alternate pathway and the relative effect of C3 spiking on complement fixation by each,

Function of the sensitized stroma used to amplify the C3a production signal from a soluble immunogen, and

Methods of quantification of the C3a peptide.

The factors influencing C3a production in MACMSA can provide C3a peptide production in the range of 20,000 to 100,000 per single epitope, average 40,000. This is a level of sensitivity that is one thousand fold (dependent on the nature of the analyte) or more times the sensitivity of standard signal amplification methods known by those skilled in the art.

Another embodiment comprises the use of a peptide with many epitopes that provides multiple Ag/Ab reactions by use of a monoclonal antibody cocktail, each antibody being specific to a different epitope on a single reporter probe. This results in fixation of an increased amount of complement, and upon activation, generation of increased numbers of C3a peptides, yielding increased levels of signal amplification from a single nucleic acid target and its reporter probe. Alternately, multiple reporter probes could be used to provide an increased C3a production and an enhanced signal amplification effect.

One embodiment of the present invention that provides enhanced complement fixation by a reporter oligonucleotide is to use an immunogenic peptide with multiple epitopes (antigenic sites) conjugated to an oligonucleotide as a reporter probe. Since an epitope is usually defined by a 3-4 amino acid region then a 40-50 amino acid peptide may bind up to 10 monoclonal antibodies each with differing specificities to the reporter peptide. Such oligonucleotides that can be modified by attachment of proteins are known by those skilled in the art.

Another preferred embodiment performing signal amplification from a single epitope on a reporter probe has the ability to generate tens of thousands of signals (ICPs) per single epitope.

This embodiment will be referred to as the membrane assisted complement mediated signal amplification or MACMSA.

Signal Amplification Generated by a Single Complement Fixing Epitope on a Reporter Probe,—an Immunogen

Antigen/antibody interactions are known to fix and activate the classical complement pathway and result in production of the ICPs discussed herein. Preferably, signal amplification from a single antigenic epitope is realized by employing multiple IgG molecules in close proximity at the Ag/Ab binding site. Also preferred is to have a lipid membrane available in close proximity to support the signal amplification effect of the complement cascade, namely enhanced ICP production. Any method may be used by those skilled in the art to provide these requirements; however, a preferred embodiment follows.

Step I:

In the embodiment depicted in FIG. 7, the dinitrophenol (DNP) antigenic epitope is conjugated at any position on a reporter oligonucleotide. Preferably, the addition is at the time of oligo synthesis and not a post-synthesis modification. The epitope is present on the phosphoramodite to assure its placement on the oligonucleotide, however, the epitope can be added by any other method such as conjugation of biotin to the phosphoramodite followed by post-hybridization reaction with a streptavidin-epitope conjugate.

Step II:

In the TPA embodiment, the reporter probe is added to a stationery or fixed protected nucleic acid sequence (PNAS) and is located on a microtiter plate wall or magnetic bead surface or any solid support.

Step III:

The requirement for a lipid membrane to support the maximal signal amplification effect can take on many forms. Plates or magnetic beads can be lipid coated, and antibody linked. The preferred embodiment of MACMSA is the use of sensitized red blood cell stroma that may be produced by any method known to those skilled in the art; however, a preferred method for producing sensitized RBC stroma follows:

Production of Sensitized RBC Stroma

A: Generate an IgG antibody pair-each antibody having different specificities: One IgG is an IgG anti D monoclonal antibody used to attach the antibody pair to the RBC surface that does not fix complement. The second IgG is an IgG anti-DNP monoclonal antibody used to bind the DNP epitope and fix and allow activation of complement with ultimate C3a production.

B: Wash red blood cells carrying the Rh determinants on the cell membrane. The Rh determinant was chosen due to the fact that the D/anti D complex is known classically to not fix complement. Any other Ag/Ab pair functioning similarly could also be employed.

C: Sensitize the RBC cells with the IgG antibody pair, forming the Ag/Ab complex that brings the IgG anti-DNP in close proximity to the red blood cell membrane lipid surface.

D. Wash the sensitized RBCs.

E. Lyse the sensitized RBCs in a hypotonic solution resulting in RBC lysis (the resulting membrane material is referred to as stroma)

F. By centrifugation, separate out the stroma and wash to remove RBC contents such as hemoglobin and proteins.

G. Resuspend the sensitized stroma in a suitable buffer to add an aliquot to a tube containing the dissociated reporter probe with the DNP antigenic epitope.

Step IV:

Addition of fresh complement and cofactors allows increased ICP production from the reporter probe DNP/anti-DNP sensitized RBC stroma interaction.

Step V:

Assay for C3a peptide production by use of any procedure known by those skilled in the art, for example:

A) ELISA (for example, sandwich) capture and reporter C3a monoclonals are necessary

B) Sensitized RBC lysis RBC-IgG anti-D—Ig anti-C3a lysis observed.

Signal Amplification Generated by a Single Complement Fixing Molecule on a Reporter Probe: a Polysaccharide

Polysaccharides and other materials are known to activate complement by the alternate pathway and result in production of the ICPs. The signal amplification effect of the present invention can be realized if the alternate pathway is functional. Whatever signal is generated by a single polysaccharide moiety can be multiplied by tethering a complex carbohydrate chain of many molecules that activate complement via the alternate pathway. This chain can be any material or polymer known to those skilled in the art that fulfills the necessary criteria and can include the use of a glucan, peptidoglyan, or complex polysaccharide.

In the case of a peptidoglyan linkage to a reporter probe, both the classical and alternate complement activation pathways may both be simultaneously used to fix and activate complement due to the complexation of polysaccharides (n-acetyl glucosamine and n-acetyl muramic acids) that have peptide linkages thereby activating both pathways. A synergism may be seen by this use of a mixed epitope in the fixation and activation of complement for signal amplification.

Signal Amplification Generated by a Single Molecule that Binds to the Reporter Probe: Histones and Similar Protein Molecules Having Affinity for the Reporter Oligonucleotides

A region can be constructed on any reporter probe that has affinity for any molecule or molecules that after binding will fix and activate complement.

To repeat, the following materials activate and fix complement:

Protein and antibody that fix C1 (classical pathway):

Antigen/Antibody complexes

For example: the following proteins and their specific antibodies

Histones,

Repressor proteins (operon regulation of transcription and translation etc.)

Materials that do not fix Cl and activate the alternate complement pathway

Particulate polysaccharides

Particulate lipopolysaccharides

Endotoxin

Trypsin-like enzymes

Antigen/antibody complexes IgA and IgG4 that do not fix complement

Any pair of molecules (affinity molecules) that appear on the reporter probe and its affinity pair may fix and thereby activate complement.

Some examples or embodiments are shown in Table 2.

TABLE 2

| | | Moiety | | |
|---|---|---|---|---|
| Reporter | Comple- | Fixing | Complement | Signal |
| Conjugated | mentary | and/or | Pathway | Amplification |
| Moiety | Molecule | Complement | Involved | Strategy |
| c-myc | IgG Anti | IgG Anti | Classical | CMSA or |
| Peptide | c-myc | c-myc | | MACMSA |
| Unique DNA | Histone | IgG Anti | Classical | CSMSA or |
| sequence | | Histone | | MACMSA |
| Unique DNA | Repressor | IgG Anti | Classical | CMSA or |
| sequence | Protein | Repressor | | MACMSA |
| | | Protein | | |
| Zymogen | Zymogen | Activated | Alternate | SLESA |
| Activation | | Zymogen | | ZMSA-1 and |
| Molecule | | | | ZMSA-2 |
| Carbo- | Properdin | Complement | Alternate | SLESA |
| hydrate | | Activation | | |

This invention is further illustrated by the following examples of diagnostic assays employing CMSA and MACMSA, which are not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims.

Aflatoxin Example

Use of the SLESA embodiments referred to as CMSA and MACMSA can be demonstrated for the ultra-sensitive detection of Aspergillus and the mycotoxins aflatoxin (AFB1). Aflatoxins are highly toxic and carcinogenic factors produced by mold contamination of soil-contacted foodstuffs such as peanuts and tobacco. They are usually produced by Aspergillus flavus and Aspergillus parasiticus and have been characterized as highly unsaturated molecules with a coumarin nucleus.

Aflatoxin B1 and G1 are the parent compounds and are potent carcinogens and have been shown to exert their carcinogenic effect by interaction with cellular nucleic acids (via adduct formation and base change). Aflatoxin B1 has been shown to suppress DNA, RNA and protein synthesis in rat liver cells. These mycotoxins, upon activation have been also shown to mutate both the p53 tumor suppressor gene as well as the K-ras genes. These mutations (guanine and cytosine transitions) implicate these mycotoxins as the causal agent in many human cancers, such as breast, colon, lung, pancreatic and others.

The mechanism of aflatoxin B 1 reaction is through the formation of DNA adducts supported by the active mode of transport of extracellular toxin into eukaryotic cells, probably mediated by its lipid nature. Similarly, liposomes themselves, lipoid in nature, are afforded rapid uptake through the cell membrane.

Processes and strategies are continually being developed that will reduce the amount of aflatoxin in the consumed product; however, the inability to sensitively detect very low levels of mycotoxin prove the limiting factor in attempts to improve the safety for use of the ingested foodstuff.

Currently, assays for AFB1 are accomplished by chromatography, including high-pressure liquid chromatography (HPLC), reversed-phase liquid chromatography, thin-layer chromatography, adsorption chromatography, immunoaffinity chromatography, gas chromatography; enzyme-linked immunoadsorbent assay (ELISA), fluorescent immunoassay, radioimmunoassay; spectroscopy, including mass spectroscopy, infrared spectroscopy, raman spectroscopy, packed-cell fluorescent spectroscopy; polymerase chain reaction (PCR), supercritical fluid extraction, bio-luminescence, chemical luminescence, and combinations thereof. Fluorescent immunoassay is a presently preferred best mode for assaying for aflatoxin on tobacco with a lower limit of sensitivity of parts per billion (trillions of molecules remain undetectable in the final processed material).

All of these above diagnostic detection techniques lack sensitivity leading to the generation of false negative diagnostic results. These assays currently offer sensitivities no less than parts per billions, meaning that even at the lowest detection level of these toxins very high numbers of molecules still remain present to achieve DNA adduct status in the tobacco user and pre-dispose him/her to a number of cancers.

The aflatoxin B1 presence in tobacco provides a major health risk for users that have been recognized. Attempts have been made to reduce and limit its presence and have been met with strong criticism due to the inability to determine its presence with high sensitivity.

Currently, FDA does not regulate AFB1 levels but does place limits of mold infection of raw tobacco to 300 parts per billion. With the knowledge that production of a single guanine or cytosine transition can predispose an individual to cancer, due to a germ cell mutation, the burden is upon diagnostics to sensitively detect the presence of aflatoxin B1 at much lower levels than is currently attainable. This increased sensitivity coupled with any effective tobacco treatment process to eliminate aflatoxin B1 can result in production of a tobacco product with much reduced risk of cancer production, a “safe” tobacco.

A technique, discussed herein, called Membrane Associated Complement Mediated Signal Amplification (MACMSA) has been developed for the detection of soluble proteins, lipids, polysaccharides, and lipopolysaccharides in solution.

The method relies upon the presence of an antigenic epitope on the molecule and a monoclonal antibody specific to this epitope, both currently available for the AFBI molecule. This interaction (antigen/antibody complex) will fix and permit complement activation, and the activation will be amplified by the presence of a lipid substrate, in this case, the sensitized RBC stroma. Again as described, complement fixation and activation will be monitored by C3a peptide production and its quantification, also herein described. This involves the classical complement fixation pathway.

Similarly, the presence of Aspergillus species organism producing the AFB1 toxin can be detected present in very low copy numbers in tobacco early in its processing. This is accomplished through Complement Mediated Signal Amplification (CMSA) and involves the alternate complement fixation pathway, namely the interaction of the molds cell surface polysaccharides and lipopolysaccharides with complement Factor B, Factor D, and properdin. No complement fixation occurs, but again complement activation occurs and can be monitored by C3a peptide production and its quantification, also herein described.

Utilizing CMSA and MACMSA, one can configure ultra-sensitive diagnostic tests to follow the tobacco from its start through each stage of its processing and resulting in the production of a tobacco/end product that is essentially devoid of AFB1.

Table 3 presents a diagnostic scheme for Aspergillus species assay and soluble AFB1 assay through the tobacco processing procedure.

TABLE 3

| | | | Each | QC |
|---|---|---|---|---|
| Test | | | Process | Volitilization |
| Material | Raw Tobacco | | Step | Testing |
| Analyte | ASPER- | SOLUBLE | SOLUBLE | SOLUBLE |
| | GILLUS SP. | AFB1 | AFB1 | AFB1 |
| Diagnostic | CMSA | MACMSA | MACMSA | MACMSA |
| Process | Alternate | Classical | Classical | Classical |
| | Complement | Complement | Complement | Complement |
| | Fixation | Fixation | Fixation | Fixation |
| | Pathway | Pathway | Pathway | Pathway |
| Theoretic | FEW | FEW | FEW | FEW |
| Sensitivity | MICRO- | MOLE- | MOLE- | MOLE- |
| Levels | ORGANISMS | CULES | CULES | CULES |
| | (10 or more) | (10 or more) | (10 or more) | (10 or more) |
| Volume of | NO | NO | NO | NO |
| Batch | LIMIT- | LIMIT- | LIMIT- | LIMIT- |
| Aliquot | ATION | ATION | ATION | ATION |
| Tested | | | | |
| Non-specific | NONE | NONE | NONE | NONE |
| Signal | | | | |
| Background | | | | |

The following are the steps that comprise the quantitative assay for the organism that is present that produces the toxin. Any toxin producing organism known can be similarly detected.

Quantitative and Automated Raw Tobacco Assay for Aspergillus Species Organisms: C3a Sandwich ELISA

Step I:

Prepare batch homogenate for testing in buffer in a microtiter plate well.

Step II:

Add magnetic beads to the well coated with a material specific for fungal cell walls, as opposed to other microbes (differential binding of intact fungi) and mix and incubate for optimum time and temperature.

Step III:

Remove the beads, wash, and place in a new plate well.

Step IV:

Add fresh complement and mix.

Step V:

Incubate at room temperature for an optimized time.

Step VI:

Remove the magnetic beads and place the supernate in a new well, to assay for C3a peptides generated, containing magnetic beads coated with the IgG anti C3a capture monoclonal antibody.

Step VII:

Wash the magnetic beads and place them in a new plate well.

Step VIII:

Add to the well IgG anti C3a reporter monoclonal antibody conjugated with an enzyme such as alkaline phosphatase and mix.

Step IX:

Wash the magnetic beads to remove unbound enzyme and place the beads into a new plate well.

Step X:

Add the 1,2 dioxetane chemiluminescent substrate and incubate at optimal time and temperature.

Step XI:

Quantify the light produced.

The following are the steps that comprise the ultra-sensitive assay for the presence of the soluble AFB1 aflatoxin.

It is important to herein note that any toxin or carcinogen known can be similarly assayed such as the most widely studied and suspected environmental carcinogens in lung cancer: polycyclic aromatic hydrocarbons (PAHs) including benzo(a)pyrene (BP) and 4-(methylnitrosoamino)-1-(3-pyridyl)-1-butanone (NNK), along with the AFB1, by a similar method.

Interestingly, all these and other carcinogens and teratogens form adducts with specific DNA bases, a major factor exploited to allow its sensitive extraction and isolation from solution in vitro. Furthermore, all the above hydrocarbons are proven to cause specific mutations to the p53 tumor suppressor and K-ras genes.

Quantitative and Automated Tobacco Processing Assay for Soluble AFB1: Capture Strategy One—DNA Adduct Formation

Step I:

Prepare a batch homogenate for testing of the presence of AFB1 in buffer and place in a microtiter plate well.

Step II:

Add magnetic beads coated with poly G•poly C duplex DNA (stable duplex) to allow adduct formation by soluble AFB1 molecules in the solution processing sample.

Step III:

Incubate at conditions favorable to formation of the adduct to bind soluble AFB1 present to the GC duplex on the magnetic beads.

Step IV:

Wash the magnetic beads and place them in a new plate well.

Step V:

Add sensitized RBC stroma (sensitized with antibody pair: IgG anti Rh-IgG anti AFB1).

Step VI:

Incubate at conditions favorable to formation of the AFB1 adduct/anti AFB1 red blood cell membrane complex.

Step VII:

To the same plate well add fresh complement and incubate at room temperature to allow production of C3a peptides.

Step VIII:

Remove the magnetic beads and transfer the remaining supemate to another plate well containing magnetic beads coated with IgG anti C3a capture monoclonal antibody and mix.

Step IX:

Remove the magnetic beads and wash to remove non-specific material, and transfer to another plate well.

Step X:

To the well, add IgG anti C3a reporter monoclonal antibody conjugated with AP, mix and incubate an optimal time.

Step XI:

Wash the magnetic beads to remove non-specific enzyme and

Step XII:

Add the 1,2 dioxetane chemiluminescent substrate to the well and incubate at an optimum time and temperature.

Step XIII:

Quantify the light produced and confirm target presence.

Quantitative and Automated Tobacco Processing Assay for Soluble AFB1: Capture Strategy Two—Affinity Molecule Association

Step I:

Place batch homogenate for testing in a microtiter plate well.

Step II:

Add magnetic beads to the well coated with a material (lipophilic or other) that binds to AFB1 or other lipid toxins.

Step III:

Remove magnetic beads, wash to get rid of non-specific material, and place beads in another plate well.

Step IV:

Add sensitized RBC stroma (sensitized with antibody pair—IgG anti D—IgG anti AFB1) to the well.

Step V:

Incubate at conditions favorable to formation of the AFB1/anti AFB1 complex, optimal time and temperature.

Step VI:

Add fresh complement to the well and incubate at room temperature to allow production of C3a peptides

Step VII:

The magnetic beads are removed and the supernate is placed in another plate well to which is added magnetic beads, coated with IgG anti C3a capture monoclonal antibody, to capture C3a produced and mix.

Step VIII:

Wash the magnetic beads to remove non-specific material and place the beads in a new plate well.

Step IX:

Add IgG anti C3a reporter monoclonal antibody conjugated with AP to the beads and mix.

Step X:

Wash the magnetic beads to remove unbound conjugate and place in a new plate well.

Step XI:

Add the 1,2 dioxetane chemiluminescent substrate to the well and incubate at optimal time and temperature to generate light.

Step XII:

Quantify the visual light produced denoting target or AFB1 presence.

Quantitative and Automated Tobacco Processing Assay for Soluble AFB1: Capture and Assay Strategy Three—Sensitized RBC Lysis (Sensitized with the Ab Pair IgG Anti D—IgG ANTI AFB1)

Step I:

Place batch homogenate for testing in microtiter plate well.

Step II:

Remove particulate material by filtration (passive) through a membrane, gravity driven.

Step III:

Add RBC sensitized cells (anti IgG anti D—IgG anti AFB1) to clear filtrate and add fresh complement.

Step IV:

Monitor RBC lysis spectrophotometrically.

This assay may be of value in the early processing steps where AFB1 molecules range in the multiple trillions.

In this example ZMSA can be fully functionally substituted for the CMSA and MACMSA process in terms of generation of target signal. Any technology that functions to similarly generate this highly important signal can be interchanged for assays in all sample areas.

This illustrates the ability of the interchangeable use of these interactive processes and embodiments.

Use of a Zymogen Conversion Reaction (ZMSA) in Single Nucleotide Polymorphism (SNP) Scoring in mRNA RP-TFO SNP Analysis

mRNA SNP analysis offers the benefit of SNP analysis in pathogen infections and those situations where the gene containing the SNP is constitutitive or constantly expressed. Furthermore, mRNA SNP analysis negates the interference of the non-coding pseudogene on the SNP scoring result.

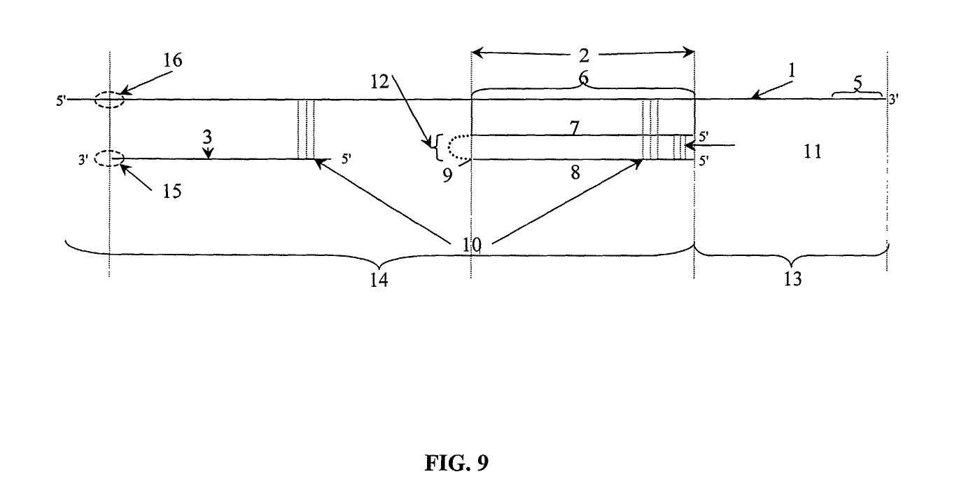

FIG. 9 depicts the embodiment of mRNA RP-TFO SNP analysis using the zymogen conversion reaction (trypsinogen→trypsin). mRNA RP-TFO is presented in related documents and the SNP scoring may be accomplished by any number of methods known to those skilled in the art, such as pyrosequencing, dideoxynucleoside terminator, single base addition, and zymogen conversion, depicted herein.

The structures presented in FIG. 9 are as follows:

1. the target mRNA, this is the analyte to be detected;

2. the PNAS, the protected nucleic acid sequence is produced by RP-TFO protection of the target region of the mRNA analyte of interest;

3. the PIP, the position identifier probe is a duplex forming oligonucleotide with specificity to the protected target region, 5′ to the RP-TFO formation site;

4. the 5′ cap, the 5′ ecapped end of the mRNA target analyte;

5. the 3′ poly A end, the 3′ poly A of the mRNA target analyte;

6. the polypyrimidine region, the PNAS formation site on the mRNA target analyte, see related documents;

7. the polypyrimidine of the RP-TFO, see related documents;

8. the polypurine region of the RP-TFO which contains 8 amino substituted purines, see related documents;

9. the biotin, used as a capture molecule, one half of the biotin/streptavidin affinity pair;

10. the hydrogen bonding, present between the polypyrimidine and polypurine strands;

11. the Hoogsteen bonds, present between the two strands of the RP-TFO, see related documents;

12. the linker and site of the reversal of polarity of the RP-TFO oligonucleotide;

13. the region of the MRNA target that will be degraded by the exonuclease selected; degradation proceeds 3′→5′;

14. the RP-TFO protected region of the target mRNA, see related documents;

15. the site of single base addition;

16. the SNP score site.

FIG. 9 represents mRNA RP-TFO SNP analysis. The following steps represent this procedure.

Step I.

Isolation of the mRNA method by any known to those skilled in the art.

Care must be taken to use universal mRNA handling procedures to avoid target destruction by environmental ribonucleases. The pH should be kept low due to the lack of activity of these non-specific environmental ribonucleases at acidic pH.

Step II.

Hybridize with a biotinylated RP-TFO with target specificity and two 5′ ends. The RP-TFO has been characterized in related documents and the two 5′ ends pose no substrate for single base addition (3′ requirement with a 5′ overhang opposite).

Step III.

Wash

Wash to remove unbound RP-TFO (Biotin) and similarly keep wash buffers at lower pH.

Step IV.

Add a 3′→5′ exonuclease

The exonuclease (3′→5′) is added to degrade all non-specific mRNA targets as well as the 3′ end of the target mRNA (from the 3′ end to the RP-TFO binding site) as per related documents. The pH is dependent upon the pH range for enzyme activity (˜pH 7.2-7.6).

Step V.

Add a streptavidin (SA) coated magnetic bead to capture the PNAS.

It is known to those skilled in the art that a SA coated magnetic bead will capture the biotinylated PNAS, and the conditions at which this will occur (pH 7.2-7.6).

Step VI.

Wash

Wash to remove non-specific materials in buffer at neutral pH. The magnetic bead now has an attached PNAS which is the site for hybridization of the Position Identifier Probe (PIP).

Step VII.

Hybridize with a PIP

The PIP hybridizes to the PNAS on the mRNA 5′ to the RP-TFO binding site (PNAS). The PIP possesses a 3′ end adjacent to a 5′ overhang, which is the prerequisite for single base addition by DNA polymerase at this site.

Step VIII.

Wash to remove unbound PIP.

Step IX. Perform single base addition to the 3′ end of the PIP.

The 3′ end of the PIP is the only site with a 3′ end suitable for single base addition by a DNA polymerase, plus dNTP and cofactors, known to those skilled in the art. The site complementary to the single base addition site represents the SNP score.

In the ZMSA preferred embodiment of SNP scoring, the four bases are conjugated with the digoxigenin (DIG) label. The SNP sample at the point of single base addition in one embodiment is separated into 4 aliquots and only one labeled (DIG) dNTP is placed in a tube containing the sample aliquot, enzyme, and cofactors, as follows:

| SCORING | | | | |
|---|---|---|---|---|
| SAMPLE | ALIQUOT 1 | ALIQUOT 2 | ALIQUOT 3 | ALIQUOT 4 |
| dNTP-DIG | dATP-DIG | dTTP-DIG | dCTP-DIG | dGTP-DIG |
| Reaction | DNA | DNA | DNA | DNA |
| Mix | polymerase | polymerase | polymerase | polymerase |
| | cofactors | cofactors | cofactors | cofactors |

Step X.

The magnetic beads are washed to remove unbound labeled dNTPs. The pH of the wash buffer should be the same as that used in the single base addition.

Step XI.

Addition of the IgG anti-DIG/enteropeptidase conjugate

The conjugate will attach only to the single DIG conjugated dNTP that is added.

Step XII.

The magnetic bead/PNAS/PIP/conjugate complex is washed to remove unbound conjugate.

Step XIII.

Add zymogen

Trypsinogen is added to be catalytically activated by the enteropeptidase of the conjugate attached to the probe complex at the appropriate pH.

Step XIV.

Generate a signal

Add a substrate (colorimetric, fluorescent, chemiluminescent, etc.) that is modified by trypsin catalysis to produce a signal.

Step XV.

Detect signal. Signal should only be detected in one of the four identical samples. The SNP score is the base complementary to the dNTP-DIG added.

Each SNP target should generate an average of 10⁶⁻⁸ signals easily providing discriminatory signal amplification for the detection down to a single target, as opposed to the 600,000 to 1,000,000 targets currently necessary for SNP scoring.

It should be understood, of course, that the foregoing relates only to preferred embodiments of the present invention and that numerous modifications or alterations may be made therein without departing from the spirit and the scope of the invention as set forth in the appended claims.

What is claimed is:

1. A method for detecting a target analyte, comprising,
a) attaching a reporter molecule to an immobilized target analyte, wherein the reporter molecule comprises a first enzyme,
b) adding a composition comprising a first proenzyme;
c) converting the first proenzyme into an active second enzyme,
d) adding a composition comprising at least one substrate of the active second enzyme; and
e) detecting the change in the substrate due to activity of the active second enzyme.

2. The method of claim 1, wherein the method further comprises adding a second proenzyme after step b); wherein the second proenzyme is changed to an active third enzyme by the activity of the active second enzyme.

3. The method of claim 1, wherein converting the first proenzyme into an active second enzyme comprises cleavage of the first proenzyme into an active second enzyme and at least one other peptide by the active first enzyme.

4. The method of claim 3, where the detecting step comprises detection of the at least one other peptide.

5. The method of claim 1, wherein the target analyte is a nucleic acid sequence, a protein, a peptide, or a single nucleotide polymorphism, or a carbohydrate.

6. The method of claim 1, wherein the compositions comprising at least one substrate comprises proteins of the complement system.

7. A method of signal amplification, comprising,
a) hybridizing a reporter molecule to an immobilized target analyte, wherein the reporter molecule comprises a first enzyme,
b) adding a composition comprising a first proenzyme;
c) converting the first proenzyme into an active second enzyme,
d) adding a composition comprising at least one substrate of the active second enzyme; and
e) detecting the change in the substrate due to activity of the active second enzyme.

8. The method of claim 7, wherein the method further comprises adding a second proenzyme after step b); wherein the second proenzyme is changed to an active third enzyme by the activity of the active second enzyme.

9. The method of claim 7, wherein converting the first proenzyme into an active second enzyme comprises cleavage of the first proenzyme into an active second enzyme and at least one other peptide by the active first enzyme.

10. The method of claim 9, where the detecting step comprises detection of the at least one other peptide.

11. The method of claim 7, wherein the target analyte is a nucleic acid sequence, a protein, a peptide, or a single nucleotide polymorphism, or a carbohydrate.

12. The method of claim 7, wherein the compositions comprising at least one substrate comprises proteins of the complement system.
